(19) United States
(12) Patent Application Publication
(10) Pub. No.: US 2022/0023363 A1
(43) Pub. Date: Jan. 27, 2022

(54) SYSTEMS AND COMPOSITIONS FOR DELIVERING ACTIVE PLANT COMPONENTS

(71) Applicant: KINSTATE, INC., LARKSPUR, CA (US)

(72) Inventors: RONALD RICHARDS, Larkspur, CA (US); JOSHUA KIRBY, Larkspur, CA (US)

(21) Appl. No.: 17/492,071

(22) Filed: Oct. 1, 2021

Related U.S. Application Data
(63) Continuation of application No. PCT/US21/14443, filed on Jan. 21, 2021.
(60) Provisional application No. 62/964,541, filed on Jan. 22, 2020.

(51) Int. Cl. A61K 36/185; A61K 9/00; A61K 45/06

(52) U.S. Cl. CPC A61K 36/185; A61K 2236/30; A61K 45/06; A61K 9/006

(57) ABSTRACT

The invention relates to systems for recreating a broad spectrum, full flower experience of a plant in a healthy, efficient, and consistent manner via the sublingual or buccal pathways.

### CLAIM OF PRIORITY UNDER 35 U.S.C. § 119

The present application is a continuation application of International Application No. PCT/US2021/014443, filed on Jan. 21, 2021, designating the United States of America which in turn claims priority to U.S. Provisional Application No. 62/964,541, filed Jan. 22, 2020. The entire contents of each of the foregoing is hereby incorporated by reference herein.

### BACKGROUND

### Field

The invention generally relates to systems for recreating a broad spectrum, full flower experience of a plant in a healthy, efficient, and consistent manner for oral delivery to the circulatory system of a user.

### Background

Traditionally, Cannabis is consumed by smoking the flower or hash derived from the plant. Various alternative methods to consume the flower or its extracts have been in use, some for centuries while others are recent. These methods include combustion via smoking, direct vaporization of the flower, vaporization using oil derived from the flower, edibles or tinctures infused with cannabinoids from the flower, as examples. When active components of the flower are smoked or vaped, they can enter the bloodstream very rapidly. When active components are ingested orally, they typically enter the bloodstream by going through the digestive system and first pass metabolism in the liver before any beneficial effects of the active components of the flower are available to the body, which typically delays onset by 30 minutes to two hours or more.

Cannabis use has great power to provide medicinal and/or personal benefits. This is largely due to the “entourage effect” that involves the synergistic interaction of multiple active components of the flower with the receptors of the endocannabinoid system as well as associated receptors throughout the body. However, many of the established approaches to delivery of active components of the flower involve extraction and/or isolation with a focus on a single molecule, such as THC or CBD. The emphasis on a single molecule deprives the user of the benefits of the entourage effect.

The focus of this invention is to provide for delivery of a broad spectrum of active components of the Cannabis flower that offers the desirable entourage effect in a manner that provides rapid uptake into the bloodstream, without requiring inhalation such as by smoking or vaping.

### SUMMARY

Some embodiments of the invention relate to a product that permits the uptake of active Cannabis components into the blood of a user within a period of time. In some embodiments, the product can include an active component profile that is substantially similar to an active component profile of a Cannabis flower.

Some embodiments of the invention relate to a product that permits the uptake of active Cannabis components into the blood of a user within a period of time, where the product can include an active component profile that is substantially similar to an artificially designed Cannabis flower active component profile.

In some embodiments, the product is a strip, film, tablet, spray, powder or gel. The strip or film can be orally disintegrating.

In some embodiments, the period of time is less than 20 minutes. In some embodiments, the period of time is less than 10 minutes. In some embodiments, the period of time is less than 9, 8, 7, 6, 5, 4, 3, 2, or 1 minute.

In some embodiments, the product can permit oral uptake via the sublingual or buccal pathways of the user. In some embodiments, the product can permit non-pulmonary uptake of the active components into the blood.

In some embodiments, the flower is from a Cannabis strain that is a natural, virtual or synthetic strain.

In some embodiments, the active component profile can include active components selected from a cannabinoid, terpene, or flavonoid.

In some embodiments, the substantial similarity can include at least 50% of major components found in the Cannabis flower quantitatively, wherein the major components are the most abundant active components of the Cannabis flower. In some embodiments, the major components are the 10 most abundant active components of the Cannabis flower, wherein an active component is a cannabinoid, a terpene or a flavonoid. In some embodiments, the major components are the 10 most abundant cannabinoids of the Cannabis flower. In some embodiments, the major components are the 10 most abundant terpenes of the Cannabis flower.

In some embodiments, the major components can be two or three of: the 5 most abundant cannabinoids, the 5 most abundant terpenes, and the 5 most abundant flavonoids. In some embodiments, the major components can be two or three of: the 3 most abundant cannabinoids, the 3 most abundant terpenes, and the 3 most abundant flavonoids.

In some embodiments, the active components in the product have relative amounts that are within 50% of relative amounts in the Cannabis flower.

In some embodiments, the product is made of all-natural ingredients.

Some embodiments of the invention relate to a method of manufacturing any of the products disclosed herein. In some embodiments, the method can include identifying a desirable profile of active Cannabis components. In some embodiments, the methods can include providing one or more starting materials that have the desirable profile. In some embodiments, the method can include incorporating the active components with other ingredients of the product. In some embodiments, the method can include obtaining the product from the results of the incorporating step.

Some embodiments of the invention relate to a method of manufacturing any of the products disclosed herein, where the strain is a virtual strain and the method can include the steps of designing an active component profile of a Cannabis flower into a desirable profile of active Cannabis components; providing one or more starting materials that comprise the desirable profile; incorporating the active components with other ingredients of the product; and obtaining the product from the results of the incorporating step.

Some embodiments of the invention relate to a method of manufacturing any of the products disclosed herein that can include the steps of designing an active component profile of a Cannabis flower into a desirable profile of active Cannabis components; providing one or more starting materials that comprise the desirable profile; incorporating the active components with other ingredients of the product; and/or obtaining the product from the results of the incorporating step. In some embodiments, the active component profile is virtual.

In some embodiments, the method can include a step of optionally extracting active components from the starting material(s) after the step of providing one or more starting materials, wherein the extracting active components can be under parameters that substantially allow preservation or reconstitution of the chemical profile.

In some embodiments, the method can include a step of combining active components prior to the step of incorporating the active components with other ingredients of the product. “Other ingredients” can be defined as any ingredient added to produce a final product in a desired formulation. For example, gelling polymers can be added to produce a product that is a gel. For example, liquid propellants can be added to produce a product that is a spray.

In some embodiments, the starting material can include one or more of: whole flower, oil, distillate, isolate, hash, resin or rosin.

In some embodiments, the extracting step can include one or more techniques of supercritical CO₂, alcohol, propane, benzene, butane, ice water hash, chromatography, distillation and/or the like.

### DETAILED DESCRIPTION

Products offering the uptake of active Cannabis components into the blood of a user within a period of time that is less than 20 minutes are provided. The product can include a chemical profile of active ingredients that is substantially similar to a Cannabis strain or combination of strain. The product can permit non-pulmonary uptake of active Cannabis components into the blood. In some embodiments, the product is a trip, film, tablet, spray, liquid, powder, or gel.

In some embodiments, the period of time is less than 15, 10, 5, or 1 minute.

The product is formulated to incorporate the broad spectrum of active components present in specific strains of plants such as Cannabis and hemp plants and release them efficiently for precise dosing and rapid absorption into the human body in a manner that provides a broad spectrum, full flower experience, thus delivering an entourage effect similar to smoking or vaping the plant material. The terms “broad spectrum” or “full flower experience” can be defined as substantially mimicking an active component profile of a starting material. The “starting material can be flower of a Cannabis strain or a combination of Cannabis strains. A theoretical starting material can be a “virtual Cannabis flower” such that a chemical profile of components normally found in Cannabis flower—such as cannabinoids, terpenes, flavonoids, and the like—is designed with amounts or ratios of ingredients that are not known to be found in any existing Cannabis strain. By virtue of this design approach, a “virtual flower” would be the part of the designed plant where such components would be found if such plant existed. In some embodiments, this approach permits assembly of new chemotypes for desired effects in the products of the invention, without the need to engage in the longer process of multiple rounds of plant breeding, chemical testing, and selection of progeny.

Except in the context of a “virtual strain,” the use of the term “strain” herein is intended to refer to any particular genetic selection of Cannabis whose properties have been characterized, such as through a certificate of analysis. As such, a strain can be a clonally propagated selection or can be an individual selected from a pheno-hunt, or can be a member of a stable seed line. Given that some of these genetic selections can also be referred to as “varieties,” “cultivars,” “chemovars,” or “chemotypes,” the use of the term “strain” herein is intended to be inclusive of these other terms where such inclusiveness is appropriate in the context of such usage. An active component can be a cannabinoid, a terpene, or a flavonoid. The active component profile can be a cannabinoid profile, a terpene profile, a flavonoid profile, or a combination of any two or all three of said profiles. Where “profile” is used herein, it makes reference to the predominant components within a profile. Given the vast biochemical diversity of Cannabis, any complete profile may have as many as dozens of components. For practicability of the invention, it is intended to reference to a profile that includes the predominant, most abundant, of those components without necessarily requiring that all of the minor components be present. For example, consider a given cannabinoid profile that has, within the limits of detection by instruments generally used in the art, ten different cannabinoids. In this example, if the three most abundant cannabinoids combine to be over 90% of the total cannabinoids and the five most abundant cannabinoids combine to be over 95% of the total cannabinoids, then for purposes of this disclosure, the “profile” can, in some embodiments, be defined as the top three cannabinoids (representing 90% of the total cannabinoids present) or the top five cannabinoids (representing 95% of the total cannabinoids present). The same principles can be applied to a terpene profile and/or a flavonoid profile, as well as to a profile of all major actives in some combination. In this approach, a “profile” of a given component or group of components can be understood to include the specific individual components that make up at least 80%, 85%, 90%, 95%, 96%, 97%, 98%, or 99% of the total amount of such component in the flower.

“Substantially mimicking” or “substantially similar” as used herein can be defined as having at least 50% similarity to the profile found in the strain or combination of strains. “Substantial similarity” can be defined as at least 50% similarity to the profile found in the strain or combination of strains. In some embodiments, the formulation can have 50%, 55%, 60%, 65%, 70%, 75%, 80%, 85%, 90% or more percent similarity to the profile of the starting material. The percentage of the profile can be determined by weight, concentration, ratios and/or number of distinct chemical components.

In some embodiments, the active component profile includes major components of the Cannabis flower. In some embodiments, major components can be defined as, for example:
the 3 or 5 or 10 most abundant active components,
the 3 or 5 or 10 most abundant cannabinoids,
the 3 or 5 or 10 most abundant terpenes,
the 3 or 5 or 10 most abundant flavonoids,
two or three of: the 2 or 3 or 4 or 5 most abundant cannabinoids, the 2 or 3 or 4 or 5 most abundant terpenes, and the 2 or 3 or 4 or 5 most abundant flavonoids;
etc., where the specific numbers listed above are understood to be merely exemplary of certain embodiments of the invention and where other numbers can be substituted into these examples without expanding the actual scope of the invention disclosed herein.

In some embodiments, percent similarity can be defined as the percentage of the major ingredients of the starting material of a Cannabis plant, qualitatively. In some embodiments, the starting material is a Cannabis flower. For example, a product with 50% similarity of a Cannabis flower has 50% of the same major components of the Cannabis flower, qualitatively.

In some embodiments, the percent similarity is quantitative. In such embodiments, for example, a product with 50% similarity to a Cannabis flower has relative amounts of the major components that are within 50% of relative amounts in the Cannabis flower.

In some embodiments, the percent similarity is both qualitative and quantitative. The terms “qualitative” and “qualitatively, as used herein relate to the presence of components of a sample. The terms “quantitative” and “quantitatively” as used herein relate to the quantities of the components in a sample. Quantities can be expressed by weight, concentration, ratios, or any other measurement using numerical units.

The broad spectrum, full flower experience can be accomplished based on the efficiency of the extraction method utilized to extract the constituent components from one or more plant strains.

The invention offers improvements to traditional smoking and vaping means which have inherent limitations. Some of these limitations are shared and some are specific to the particular mode of consumption. For example, any attempt to deliver components via the lungs runs into an inherent limitation regarding how much of the desired components actually arrive deep enough into the lungs to be efficacious. It is generally thought that only about 15% of the active component arrive deep enough into the lungs to be absorbed into the blood stream. The rest either is imbedded along the inner walls or remains in gas phase or gaseous suspension and is ultimately exhaled with no effect. The only known solution to this problem is to engineer the aerodynamic shape of particles themselves in a manner that increases the likelihood that they will reach their intended destination deep in the lungs. This is costly and difficult to achieve given differences in the anatomy and physiology from individual to individual.

In addition, both smoking and vaping require the individual consuming the product to inhale foreign particulate matter into the lungs in the form of smoke or chemical vapor. This is generally viewed as undesirable and unhealthy and, for some uses, is an unacceptable obstacle to enjoying the benefits of Cannabis use. In some embodiments, the present invention offers solutions to these issues in the form of oral dissolving preparations which are uniquely capable of achieving the goal of delivering a broad spectrum, full flower experience in a healthy manner, and by utilizing oral pathways, such as the sublingual or buccal pathways, particularly via the oral mucosal membrane under the tongue, the full complement of components from the plant can be delivered directly to the blood stream efficiently, rapidly, precisely and consistently.

In certain embodiments, the formulations of the invention contain more than one cannabinoid, which provide an entourage effect. The invention relates to methods and products to recreate the entourage effect generally associated with different strains of the plant rather than isolating particular components from the plant to address a specific need as has been done in the art. Instead, the invention relates to recreating a broad spectrum, full-flower experience in a healthier, more efficient delivery vehicle such as, for example, via the sublingual or buccal pathways. The combined effect of the various components in the plant provide an experience that cannot be attributed to any one element present in the plant.

The chemical compositions used in the product can be based on a single strain or multiple strains. The strain on which the product is based can be a known strain, a synthetic strain, or a virtual strain. For purposes of this disclosure, a “known strain” is a strain or cultivar of Cannabis that is publicly known or publicly available, or that exists as a land race or that exists in nature, such that the known strain has a known chemical profile of active components. It is understood that such chemical profiles, also known as chemotypes, can vary with cultivation conditions and other factors. Nonetheless, any chemical profile associated with a known strain can be considered to be useful for the invention and use of any known strains chemotypic information is within the scope of the invention. When the composition is based on a known strain, the ingredients of the composition are derived from such strain such as via extraction. Likewise, the strain can be a synthetic strain. For purposes of this disclosure, a synthetic strain is a chemotype that mimics, in whole or in part, the chemotype of a known strain, where one or all of the components are not directly sourced from the known strain. Thus, a synthetic strain can be a chemotype that is essentially assembled or reconstituted based on the chemotype of a known strain. In some embodiments, chemotypic components of two or more known strains can be blended together to deliver what could be classified as a new “virtual strain.” As used herein, a “virtual strain” can be defined as a chemotypic profile that does not match that of any known strain but has been manufactured to have desired properties. Thus, for purposes of this disclosure, the term “strain” can be applied either to actual plant selections or to the chemotypic profile of an extract of such plant selections, or to a chemotypic profile reconstituted to mimic such an extract in whole or in part, or to a chemotypic profile created by combining actives in such a way as to not correspond to any known plant selection.

The chemical composition can include at least 3, 4, 5, or more active components of a Cannabis plant. The active components can be various combinations of cannabinoids, terpenes, flavonoids, any naturally occurring plant compound and/or the like. For example, in some embodiments, the chemical composition can include at least one each of a cannabinoid, a terpene and a flavonoid. The active component can be a therapeutically beneficial molecule or combinations of the same such as delta-9-tetrahydrocannabinol, delta-8-tetrahydrocannabinol, cannabinol, cannabidiol, tetrahydrocannabinolic-acid, cannabidiolic-acid, cannabigerol, cannabigerolic-acid, cannabichromene, tetrahydrocannabivarin, cannabivarin and/or any other cannabinoid, terpene, or naturally occurring plant component. In some embodiments, the component is found in Cannabis, but isolated from a non-Cannabis plant.

The cannabinoid can be, for example, one or more of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), cannabidiorcol (CBD-C1), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C4 (THCA-C4), delta-9-tetrahydrocannabinol-C4 (THC-C4), delta-9-tetrahydrocannabivarinic acid (THCVA), delta-9-tetrahydrocannabivarin (THCV), delta-9-tetrahydrocannabiorcolic acid (THCA-C1), delta-9-tetrahydrocannabiorcol (THC-C1), delta-7-cis-iso-tetrahydroc annabivarin, delta-8-tetrahydrocannabinolic acid (Δ8-THCA), delta-8-tetrahydrocannabinol (Δ8-THC), cannabicyclolic acid (CBLA), cannabicyclol (CBL), cannabicyclovarin (CBLV), cannnabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C4 (CBN—C4), cannabivarin (CBV), cannabinol-C2 (CBN—C2), cannabiorcol (CBN—C1), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR) and trihydroxy-delta-9-tetrahydrocannabinol (triOH-THC), or the like.

The terpene can be, for example, a-bisabolol, borneol, camphene, camphor, 3-carene, caryophyllene oxide, b-caryophyllene, a-cedrene, citronellol, p-cymene, eucalyptol, fenchol, geraniol, geranyl acetate, guaiol, a-humulene, isobomeol, (−)- isopulegol, limonene, linalool, menthol, myrcene, nerolidol, ocimene, phellandrene, phytol, a-pinene, b-pinene, R-(+)-pulegone, sabinene, a-terpinene, terpinen-4-ol, a-terpineol, 4-terlineol, terpinolene, valencene, or the like.

The flavonoid can be, for example, apigenin, cannflavin A, cannflavin B, cannflavin C, chrysoeriol, cosmosiin, flavocannabiside, kaempferol, luteolin, myricetin, orientin, isoorientin (homoorientin), quercetin, (+)-taxifolin, vitexin, and isovitexin, or the like.

The active component profile can be based on composition of matter which can be determined by generally recognized methods such as chromatography or mass spectrometry, light refraction and/or the like.

In some embodiments, the product can be made of entirely all-natural ingredients. As used herein, the term “all-natural” means that the ingredient does not include any synthetic or artificial components. In other embodiments, the product can include synthetic or artificial components.

Some embodiments of the invention relate to broad spectrum or full flower compositions provided by oils, distillates, or other starting materials and converting them into an efficient formulation. The chemical composition can be in the form of a strip, tablet, spray, liquid, powder, gel, or the like. The product can be orally disintegrating, wherein the product disintegrates in the mouth of a user. Methods for producing the orally disintegrating (typically sublingual) strip can be found in PCT Application No. PCT/US20/61380, entitled “ORAL STRIPS AND METHODS OF MAKING SAME,” filed on Nov. 19, 2020, which claims priority to U.S. Provisional Application No. 62/937,909, entitled “ORAL STRIPS AND METHODS OF MAKING SAME,” filed on Nov. 20, 2019; each of which is fully incorporated by reference herein.

The oils, distillates, or other starting material can be any material isolated from the plant (e.g., oil, distillate, isolates, hash, resin, rosin, etc.).

Methods of manufacturing any of the products described herein are provided.

In some embodiments, the method can include identifying and obtaining a starting plant material or a virtual starting plant material with a desirable effect profile. A desirable effect can be defined as an actual or perceived benefit to an end user or a useful feature for marketing the product. A desirable effect profile can include an effect that is a positive experience for a user that can include a specific or broad psychological effect or effects, emotional effect or effects, physical effect or effects, medicinal effect or effects or combination of any perceived or actual effects induced in human or animal subjects. Starting material can be selected or manufactured based on its popularity, desirability, name, appellation, geography of origin, smell, color, taste, or any other characteristic that would provide an actual or perceived benefit to an end user or a useful feature for marketing the product. The effect profile can be determined by analyzing an active component profile of an existing strain or a combination of profile(s) of existing strains or a new synthetic or virtual strain. The active component profile can be a chemotype profile, a cannabinoid profile, a terpene profile, a flavonoid profile, or a combination of said profiles of a strain or combination of strains. The active components of an effect profile can be selected based on physiological effects of the component on a user. The starting material can be sourced in its original whole plant biomass form, a pulverized or ground form of the plant or combination of plants, a crude or refined extract of the plant or combination of plants, specific or broad molecular fractions of the plant extract, or any combination of these deemed necessary or desirable to create the intended final product.

In some embodiments, the method can include analyzing the starting material to determine the active component profile of the plant matter. The profile can be determined by analyzing the active component profile of an existing strain or a combination of the active component profiles of existing strains or a new synthetic or virtual strain. The starting material can include one or more of oil, distillate, isolate, hash, resin, rosin, and/or the like. The active component profile can be determined through chromatography or any other standard analytical process suitable for determining the specific chemical makeup of the starting material or materials.

In some embodiments, the method can include extracting from starting material under parameters that substantially allow preservation or reconstitution of the composition profile. “Substantially” can be defined as preserving or reconstituting at least 50% of the active component profile or preserving at least 50% of the active component profile so that they can be reconstituted in the next step. The extraction step can employ supercritical or subcritical CO₂, alcohol, propane, benzene, butane, ice water hash, chromatography, distillation and/or the like. In the case of creating a virtual or synthetic strain in which a compound that is not present in the correct form in the starting material may be required to formulate a product, methods such as decarboxylation, chemical synthesis or semisynthesis, and/or the like can be used.

In some embodiments, the method can include combining the active components. The resulting extracts can be combined in a way that can result in a final composition of one or extracts or fractions that mirror the active component composition analysis of the starting material. One or more homogenization methods can be used, including mixing, blending, stirring, and/or the like.

In some embodiments, the method can include incorporating the chemical components with other ingredients into a formulation.

### Sublingual Strip

In some embodiments, the product is an oral strip. The oral strip can include, or can be manufactured with a solution including, water, saponin or a saponin-rich plant extract, one or more oils with one or more active agents, and/or one or more hygroscopic polysaccharide(s). The percent water can be 30-80%. In some embodiments, the percent water can be 52.5%-65.0%. For example, the percent water can be about 52.5, 55, 60, or 65%. The percent saponin or saponin-rich plant extract can be 0.2-10.0%. In some embodiments, the percent saponin or saponin rich plant extract can be 1.6%-3.0%. For example, the percent saponin or saponin-rich plant extract can be about 1.6, 2, 2.2, 2.4, 2.6, 2.8, or 3%. The percent oil can be 1-40%. In some embodiments, the percent oil can be 10.0%-15.0%. For example, the percent oil can be about 10, 11, 12, 13, 14, or 15%. The pullulan can be pullulan that is natural in origin and unmodified. The percent hygroscopic polysaccharide can be 1-40%. In some embodiments, the percent pullulan can be 5.0%-17.0%. For example, the percent pullulan can be about 5, 6, 7, 8, 9, 10, 11, 12, 13, 14, 15, 16 or 17%. The oral strip can be made of all-natural ingredients.

In some embodiments, the oil can be a vegetable or botanical oil. Examples of oils that can be used include, but are not limited to, hemp oil, cannabis oil, MCT oil, coconut oil, avocado oil, peppermint oil, mango oil, sunflower oil, cottonseed oil, citrus oil, safflower oil, rice bran oil, cocoa butter, palm fruit oil, palm kernel oil, soybean oil, rapeseed oil, corn oil, sesame oil, walnut oil, flaxseed oil , jojoba oil, castor oil, grape seed oil, peanut oil, olive oil, algae oil, mushroom oil, or the like, or any combination thereof.

Saponins are a subclass of terpenoids and can also be referred to a triterpene glycosides. Saponins have been reported in more than 100 families of plants and in a few marine sources such as star fish and sea cucumber. Examples of saponin-rich plant extract sources used in the invention can include, but are not limited to soap bark tree (Quillaja saponaria), Mojave yucca (Yucca schidigera), licorice (Glycyrrhiza species), ginseng (Panax species), fenugreek (Trigonellafoenum graceum), alfalfa (Medicago sativa), horse chestnut (Aesculus hippocastanum), soapwort (Saponaria officinaux), gypsophila genus (Gypsophila paniculata) and sarsaparilla (Smilax species) or other sources, and/or the like, and/or combinations of the same. Saponins of use can include, but are not limited to, soyasaponin A(1), soyasaponin A(2), soyasaponin I, soyasaponin B, deacetylated soyasaponin, acetylated soyasaponin, soyasaponin II, soyasaponin III and soyasapogenol B monoglucuronide. In preferred embodiments, the saponins of use may be present in an extract, homogenate, finely ground powder, or other derivative of plant matter, such as beans, peas, nuts or other plant parts. In some embodiments, the relative proportions of plant extracts, homogenates, finely ground powders, or derivatives can be selected to optimize the content of one or more saponins of use in the composition for oral delivery of an active agent.

The one or more active agents can be any essential plant extract that has a concentration of active agent between 25% and 99.9% of the total mass.

The polysaccharide used in the invention can be from a natural substance. Examples of such polysaccharides include, but are not limited to, polymers obtained from natural substances such as acacia gum, gum arabic, sodium alginate, casein, xanthan gum, guar gum, dextran, tragacanth gum, starch, pullulan, pectin, and/or the like.

In some embodiments, the saponin can act as an emulsifier. In such embodiments, the hydrophilic-lipophilic balance (HLB) of the saponin can be between about 8-17. For example, the HLB can be about 13.5.

In some embodiments, the strip can contain beneficial volatile organic compounds that evaporate under standard conditions if not contained in the strip. Examples of such ingredients can include, but not be limited to, the terpenes listed above as well as any other VOC capable of conferring a benefit on the user.

In some embodiments, the strip made of entirely all-natural ingredients has similar or improved physical characteristics compared to a strip made of one or more synthetic ingredients. The physical characteristic can include tensile strength, percent elongation, elastic modulus, folding endurance, thickness, disintegration time, uniformity of drug content, potency, moisture content, weight, pharmacokinetics, etc.

References that provide characteristics of systems made of synthetic ingredients include: Nayak B S, International Journal of Pharmaceutics & Drug Analysis; Vol. 5, Issue 10, 2017; 399-405; Mannila J, Järvinen T, Järvinen K, Tervonen J, Jarho P. “Sublingual administration of Delta9-tetrahydrocannabinol/beta-cyclodextrin complex increases the bioavailability of Delta9-tetrahydrocannabinol in rabbits.” Life Sci. 2006; 78(17):1911-1914; Narang N, Sharma J. “Sublingual Mucosa as a Route for Systemic Drug Delivery.” Int J of Pharm Sci, Vol. 3, Suppl 2, 2011, 18-22; and Katz M, Barr M. “A study of sublingual absorption. I. Several factors influencing the rate of adsorption.” J Am Pharm Assoc Am Pharm Assoc. 1955; 44(7):419-423. Each of the foregoing is hereby incorporated by reference in its entirety.

Some embodiments of the invention relate to methods of manufacturing an oral strip with one or more active agents. The method can include the steps of creating a stable emulsion of one or more oils containing one or more active agents in water using one or more saponins; combining the stable emulsion with a polysaccharide to create a slurry; and casting the slurry into an oral strip.

The step of creating a stable emulsion of one or more oils containing an active agent can include the use of a high-speed mixing apparatus such as an immersion blender, homogenizing wand, emulsifying mixer, a mixing apparatus that utilizes sound energy such as a sonicating wand, a mixing apparatus that utilizes high pressure energy such as a microfluidizer, and/or the like. The mixture can be prepared at room temperature. The emulsion can be mixed for a period of 2 minutes to 24 hours depending on application. In some embodiments, the method can include multiple successive mixing sessions to achieve a stable emulsion.

The step of combining the stable emulsion with pullulan to create a slurry can include maintaining both the emulsion and the polysaccharide(s) at room temperature and combining at a speed of between 30-120 RPM using a planetary mixer for 2 min-24 hrs. Alternatively, the step can include combining at a speed of between 1000-5000 RPM using an emulsifying mixer.

The step of combining the stable emulsion with pullulan to create a slurry can further include adding one or more additional ingredients. The one or more additional ingredients can include natural flavorings such as fruit and herbal extracts and/or sweeteners; natural colorings such as fruit, vegetable and/or herbal powders and/or extracts; humectant(s) such as glycerin, lecithin, beeswax, honey, aloe vera and/or other natural humectants; natural phospholipids such as sunflower lecithin, soy lecithin, egg lecithin, bovine lecithin, and/or other natural phospholipids; and natural gums including acacia gum, xanthan gum, agar, gellen gum, guar gum, locust bean gum, and/or other natural gums.

The step of casting the slurry into an oral strip can include mold casting; die casting; roll to roll casting equipment that utilizes doctor-blades, coating bars, or slot-dies; automatic and/or manual drawdown coating equipment; doctor-blade casting equipment; sputter coating equipment; electrospinning equipment; spin coating, dip coating, bar coating, spray deposition and or any method practicable for creating a film of uniform thickness on a substrate.

The oral strip can be any size or shape that would reasonably fit under a user's tongue such as a quadrilateral that is between 0.125″×0.125″-1.5″×1.5″, non-ridged shapes (such as crescent moons, horseshoes) or round shapes (such as ovals or circles). The strips may be of a thickness between 0.0001″ and 0.5″. In some embodiments, the strips can have a thickness of 0.0002, 0.0005, 0.001, 0.005, 0.01, 0.05, 0.1, or 0.25″.

In some embodiments of the invention, as the slurry is cast into strips and cured, the natural saponins stabilize the position of the oil(s) so that the homogeneity of the ingredients achieved during the mixing stage remains high and the oil(s) ultimately remain in the film.

### Further Formulations

The product can also be manufactured as a film, tablet, spray, powder or gel according to means known in the art.

The product can have a final active ingredient concentration of about between 0.01% and 35%. In some embodiments, the final active ingredient concentration can be 0.02, 0.05, 0.1, 0.5, 1, 3, 5, 10, 15, 20, 25, or 30%.

### EXAMPLES

### Example 1

A desired Cannabis strain is identified. The strain is popular with users for providing a desired effect after inhalation via smoking or vaporization. A starting material from the strain is analyzed for a cannabinoid profile and the 10 most abundant cannabinoids are identified. A product in the form of a sublingual strip is made including at least 5 of the most abundant cannabinoids. Use of the sublingual strip provides similar effects as inhalation via smoking or vaporization of the starting material.

### Example 2

A desired Cannabis strain is identified. The strain is popular with users for providing a desired effect after inhalation via smoking or vaporization. A starting material from the strain is analyzed for a cannabinoid profile and a terpene profile. The 10 most abundant cannabinoids and the 10 most abundant terpenes are identified. A product in the form of a sublingual strip is made including at least 5 of the most abundant cannabinoids and 5 of the most abundant terpenes. Use of the sublingual strip provides similar effects as inhalation via smoking or vaporization of the starting material.

### Example 3

Two desired Cannabis strains are identified. The strains are popular with users for providing a desired effect after inhalation via smoking or vaporization. Starting materials from the strains are analyzed for a cannabinoid profile and a terpene profile. The profiles are combined to obtain a virtual profile for a synthetic strain. A formulation in the form of a sublingual strip is made including the virtual profile. The sublingual strip provides superior effects compared to inhalation via smoking or vaporization of the starting material.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A product that permits the uptake of active Cannabis components into the blood of a user within a period of time; wherein the product comprises an active component profile that is substantially similar to an active component profile of a Cannabis flower, wherein the product is a strip, film, or gel; wherein the product permits oral uptake via the sublingual or buccal pathways of the user.

2. A product that permits the uptake of active Cannabis components into the blood of a user within a period of time; wherein the product comprises an active component profile that is substantially similar to an artificially designed Cannabis flower active component profile, wherein the product is a strip, film, or gel; wherein the product permits oral uptake via the sublingual or buccal pathways of the user.

3. The product of claim 1, wherein the period of time is less than 20 minutes.

4. The product of claim 1, wherein the period of time is less than 10 minutes.

5. The product of claim 1, wherein the Cannabis flower is from a Cannabis strain that is a natural, virtual or synthetic strain.

6. The product of claim 1, wherein the active component profile comprises active components selected from a cannabinoid, terpene, or flavonoid.

7. The product of claim 1, wherein the substantial similarity comprises at least 50% of major components found in the Cannabis flower quantitatively, wherein the major components are the most abundant active components of the Cannabis flower.

8. The product of claim 7, wherein the major components are the 10 most abundant active components of the Cannabis flower, wherein an active component is a cannabinoid, a terpene or a flavonoid.

9. The product of claim 7, wherein the major components are the 10 most abundant cannabinoids of the Cannabis flower.

10. The product of claim 7, wherein the major components are the 10 most abundant terpenes of the Cannabis flower.

11. The product of claim 7, wherein the major components are selected from two or three of: the 5 most abundant cannabinoids, the 5 most abundant terpenes, and the 5 most abundant flavonoids.

12. The product of claim 7, wherein the major components are selected from two or three of: the 3 most abundant cannabinoids, the 3 most abundant terpenes, and the 3 most abundant flavonoids.

13. The product of claim 1, wherein the active components in the product have relative amounts that are within 50% of relative amounts in the Cannabis flower.

14. The product of claim 1, wherein the product consists of all-natural ingredients.

15. A method of manufacturing the product of claim 1, comprising the steps of:
a. identifying a desirable profile of active Cannabis components;
b. providing one or more starting materials that comprise the desirable profile;
c. incorporating the active components with other ingredients of the product; and
d. obtaining the product from the results of the incorporating step.

16. The method of claim 15, additionally comprising the step of optionally extracting active components from the starting material(s) after the step of providing one or more starting materials, wherein the extracting active components is under parameters that substantially allow preservation or reconstitution of the chemical profile.

17. The method of claim 15, additionally comprising the step of combining active components prior to the step of incorporating the active components with other ingredients of the product.

18. The method of claim 15 wherein the starting material comprises one or more of whole flower, oil, distillate, isolate, hash, resin or rosin.

19. The method of claim 15, wherein the extracting step comprises one or more of supercritical CO₂, alcohol, propane, benzene, butane, ice water hash, chromatography, distillation and/or the like.

20. A method of manufacturing the product of claim 2, wherein the strain is a virtual strain, the method comprising the steps of:
a. designing an active component profile of a Cannabis flower into a desirable profile of active Cannabis components;
b. providing one or more starting materials that comprise the desirable profile;
c. incorporating the active components with other ingredients of the product; and
d. obtaining the product from the results of the incorporating step.

21. The method of claim 20, additionally comprising the step of optionally extracting active components from the starting material(s) after the step of providing one or more starting materials, wherein the extracting active components is under parameters that substantially allow preservation or reconstitution of the chemical profile.

22. The method of claim 20, additionally comprising the step of combining active components prior to the step of incorporating the active components with other ingredients of the product.

23. The method of claim 20, wherein the extracting step comprises one or more of supercritical CO₂, alcohol, propane, benzene, butane, ice water hash, chromatography, distillation and/or the like.
